(19) United States
(12) Patent Application Publication
(10) Pub. No.: US 2023/0320812 A1
(43) Pub. Date: Oct. 12, 2023

(54) MEDICAL DEVICE SUPPORT DEVICES

(71) Applicant: ABIOMED, Inc., Danvers, MA (US)

(72) Inventors: Glen FANTUZZI, Danvers, MA (US); Jonathan BARRY, Danvers, MA (US); Andrea MERIN, Danvers, MA (US)

(73) Assignee: ABIOMED, Inc., Danvers, MA (US)

(21) Appl. No.: 18/131,918

(22) Filed: Apr. 7, 2023

Related U.S. Application Data
(60) Provisional application No. 63/328,642, filed on Apr. 7, 2022.

(51) Int. Cl. A61B 90/50; F16M 11/22

(52) U.S. Cl. CPC A61B 90/50; F16M 11/22; F16M 2200/028; F16M 2200/08

(57) ABSTRACT

Support devices and/or support blocks are provided that support medical devices, such as access devices, at an desired orientation relative to a patient's body. Support blocks may include a bottom surface and a top surface, where the top surface may be configured to be substantially at an angle (such as 5-30 degrees) relative to the bottom surface. The top and/or bottom surface may have a connection mechanism configured to allow the support block to connect with at least one additional support block. The connection mechanism may be, e.g., a protruding element that may be configured to interface with a receiving slot on the at least one additional support block, a receiving slot that may be configured to receive a protruding element from the at least one additional support block, a magnet that may be configured to interface with a magnet or ferromagnetic material on the at least one additional support block, and/or one or more protrusions or depressions that may be configured to form an interference fit with one or more protrusions or depressions on the at least one additional support block.

### CROSS-REFERENCE TO RELATED APPLICATIONS

The present application claims priority to U.S. Provisional Patent Application 63/328,642, filed Apr. 7, 2022, which is incorporated by reference herein in its entirety.

### TECHNICAL FIELD

The present disclosure is drawn to support structures for medical devices that may extend outward from a patient's body.

### BACKGROUND

In clinical settings, medical devices may be inserted into a patient at an access point via an access device (e.g., an introducer sheath). In some instances, a portion of such medical devices may extend outward from the body. In some cases, this may occur while the patient is on medical support for an extended period, e.g., 6 hours or more. As is known the medical device may be fixated to the patient (e.g., via one or more suture pads).

### BRIEF SUMMARY

In some embodiments, a support block is disclosed. The support block may include a bottom surface and a top surface. The top surface may be configured to be substantially at an angle (such as 5-50 degrees) relative to the bottom surface. As will be appreciated, the angle of the support block may be configured to match a desired support angle of a medical device and/or access device to be maintained at an access location. The top surface may be connected to the bottom surface via a front surface, two side surfaces, and a rear surface. The rear surface may have a larger surface area than the front surface. The top and/or bottom surface may have a connection mechanism configured to allow the support block to connect with at least one additional support block. The connection mechanism may be, e.g., a protruding element that may be configured to interface with a receiving slot on the at least one additional support block, a receiving slot that may be configured to receive a protruding element from the at least one additional support block, a magnet that may be configured to interface with a magnet or ferromagnetic material on the at least one additional support block, and/or one or more protrusions or depressions that may be configured to form an interference fit with one or more protrusions or depressions on the at least one additional support block.

In some embodiments, the support block may include a silicone or a thermoplastic polyurethane. In some embodiments, the support block may include a rigid material. In some embodiments, the support block may be formed of an open or closed cell foam. In some embodiments, the support block may include an antiseptic filler, such as silver.

In some embodiments, the support block may include an angle-matching device coupled to the bottom surface, the angle-matching device configured to be sutured to the patient. In some embodiments, the support block may be configured to be sutured to the patient. In some embodiments, the support block may include an adhesive layer on a bottom surface. In some embodiments, the bottom surface of the block also may be formed of a material (or coating) with a high coefficient friction (e.g., a “tacky” material) such that the position of the block on the patient may not move substantially once placed by a clinician.

In some embodiments, the top surface may be contoured to receive an extracorporeal portion of an access device. In some embodiments, the top surface may include a depression configured to contact and partially surround a portion of an extracorporeal portion of an access device and/or medical device. In some embodiments, the bottom surface of the support block may be contoured to fit a location on the patient and/or a body type of the patient, such as an anterior (e.g., upper anterior) surface of a patient's thigh or an upper chest region (e.g., near the subclavian or auxiliary artery). In some embodiments, the rear surface may include a groove configured to provide access to a finger tip.

In some embodiments, a support device may be provided that includes a collar portion configured to at least partially surround a hub, catheter, cannula, and/or stent, and an adjustable portion hingedly coupled to the collar portion.

In some embodiments, a support device may be provided that includes an angle-matching device configured to be sutured to the patient, and an adjustable arm configured to be coupled to the angle-matching device and a sheath hub, catheter, cannula, and/or stent.

In some embodiments, a system may be provided. The system may include an access device having an extracorporeal portion extending away from a patient and one or more support blocks or support devices as disclosed herein. The one or more support blocks and/or support devices may be configured to allow the extracorporeal portion to maintain a desired orientation relative to the patient. In some embodiments, the access device may be a catheter. In some embodiments, the access device may be an introducer sheath. In some embodiments, the system may include 2-5 support blocks.

In some embodiments, each support block is configured to have a different angle (e.g., the angle of the top surface relative to the bottom surface). In some embodiments, each support block is configured to have the same angle.

In some embodiments, a bottom surface of one support block may be contoured to fit a location on the patient and/or a body type of the patient. In some embodiments, a top surface of one support block may be contoured, or may contain a depression.

In some embodiments, each support block may have a top surface and/or bottom surface that is configured to physically interlock with at least one other support block. In some embodiments, each support block may have a top surface and/or bottom surface that is configured to interact magnetically with at least one other support block. In some embodiments, each support block may have a top surface and/or bottom surface that is configured to connect to at least one other support block via an interference fit.

In some embodiments, a kit may be provided. The kit may include an access device and one or more support blocks or support devices as disclosed herein.

The one or more support blocks may be configured to allow an extracorporeal portion of the access device to maintain a desired orientation relative to a patient, each support block comprising a bottom surface and a top surface substantially at an angle relative to the bottom surface. The kit may include an angle-matching device coupled to a bottom surface of one of the one or more support blocks, the angle-matching device configured to be sutured to the patient.

In some embodiments, the kit may include 2-5 support blocks. In some embodiments, each support block is configured to have the same angle. In some embodiments, each support block is configured to have a different angle.

In some embodiments, a bottom surface of one support block may be contoured to fit a location on the patient and/or a body type of the patient. In some embodiments, a top surface of one support block may be contoured, or may contain a depression.

In some embodiments, each support block may have a top surface and/or bottom surface that is configured to physically interlock with at least one other support block. In some embodiments, each support block may have a top surface and/or bottom surface that is configured to interact magnetically with at least one other support block. In some embodiments, each support block may have a top surface and/or bottom surface that is configured to connect to at least one other support block via an interference fit.

### BRIEF DESCRIPTION OF DRAWINGS

FIG. 1 is an illustration of an embodiment of a support block.

FIG. 2 is an illustration of an embodiment of a support block.

FIG. 3 is an illustration of an embodiments of a system that includes multiple support blocks.

FIG. 4 is an illustration of an embodiment of a support block with a contoured top surface.

FIG. 5 is an illustration of an embodiment of a support block with a contoured bottom surface.

FIGS. 6A and 6B are illustrations of embodiments of support blocks.

FIG. 7 is an illustration of an embodiment of a support block.

FIGS. 8-10 are illustrations of embodiments of a support device.

FIG. 11 is a flowchart of an embodiment of a method.

FIG. 12 is a side view of an access device according to another embodiment.

FIG. 13 is a rear view of a portion of the access device of FIG. 12.

FIG. 14 is a rear view of a portion of an access device according to another embodiment.

FIG. 15 is a side view of an access device according to another embodiment.

FIG. 16 is a top view of the access device of FIG. 15.

FIG. 17 is a top view of the access device of FIG. 15, with support arms in a first position.

FIG. 18 is a top view of the access device of FIG. 15, with support arms in a second position.

### DETAILED DESCRIPTION

As is known, medical devices may be inserted into a patient at an access point via an access device (e.g., an introducer sheet), and a portion of such medical devices may extend outward from the body. In some cases, this may occur while the patient is on support for an extended period, e.g., 6 hours or more.

In some instances, medical devices may have long, rigid lengths that extend outward from the body. This may create a lever arm that, when compressed down to the skin or body, can cause lifting or tenting of the vasculature under the subcutaneous tissue. This lifting or tenting of the vasculature may lead to bleeding and subsequent patient discomfort or harm.

Existing solutions are typically built into the catheter or introducer devices themselves, such as by trying to ensure that the stiffness of the catheter shafts is low. In some embodiments, if the device shafts are very soft, the rigid hubs of the products can be pushed down to the skin without transmitting lift to the subcutaneous vessel. Other solutions may involve clinicians placing folded up gauze under the access device to maintain a desired position. The inventors have recognized that such arrangements do always provide a satisfactory solution.

Accordingly, in view of the above, the inventors have recognized the benefit of a support system configured to maintain a desired support angle between a medical device exiting a patient at an access location. As described herein, the support system may be configured to maintain a desired support angle between an access device and the patient. In some embodiments, the support system may include one or more support blocks and/or one or more support arms.

As described herein, the desired support angle of the medical device may include the naturally protruding angle of the medical device (e.g., the angle of the medical device exiting the access device, such as the introducer or catheter, relative to the patient), allowing for the system to be stabilized and to minimize or prevent the compression of the device down to the skin. Since patients may have varying anatomy based on size, weight, height, and general access site, the proposed embodiments allow for a simple way for practitioners to adjust and accommodate the true angle of the specific medical device (e.g., a catheter) relative to any patient anatomy.

Once a medical device is placed, and as it is protruding outward from the skin, embodiments of the present disclosure can be moved between the device and the patient's body to maintain the protruding angle. For example, in some embodiments, the support system may include one or more support blocks that are slid under the device and between the device and the patient's body (e.g., anterior leg). If the angle is shallow the system allows for the user to “select” or adjust by removing a stackable setup.

In some embodiments, by ensuring the device is stable at a natural angle, the bleeding due to tenting while the patient is in the ICU for long durations may be minimized or even prevented.

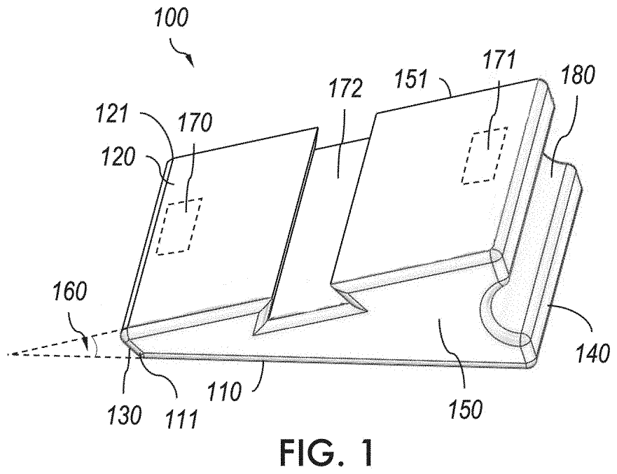

Turning now to the figures, FIG. 1 illustrates a support block 100 according to one embodiment of the present disclosure. As show in this view, the support block may include a bottom surface 110 and a top surface 120. In some embodiments, the support block may include a front surface 130, a rear surface 140, and two side surfaces 150, 151. As will be appreciated, the bottom surface 110 may be configured to be placed against a patient. In such embodiments, a medical device (and/or access device) may rest stably against at least a portion of the top surface 120 of the block.

In some embodiments, the top surface 120 is configured to be substantially at an angle 160 relative to the bottom surface. In some embodiments, the angle may be between 5 degrees and 60 degrees. In some embodiments, the angle may be between 5 degrees and 30 degrees. The angle may be formed by taking an imaginary plane parallel to the front-most portion 121 of the top surface 120, ignoring any chamfers, bezels, or rounded edges, and measuring the angle at which it intersects an imaginary plane parallel to the front most portion 111 of the bottom surface 110, ignoring any chamfers, bezels, or rounded edges.

In some embodiments, the rear surface 140 may have a larger surface area than the front surface 130.

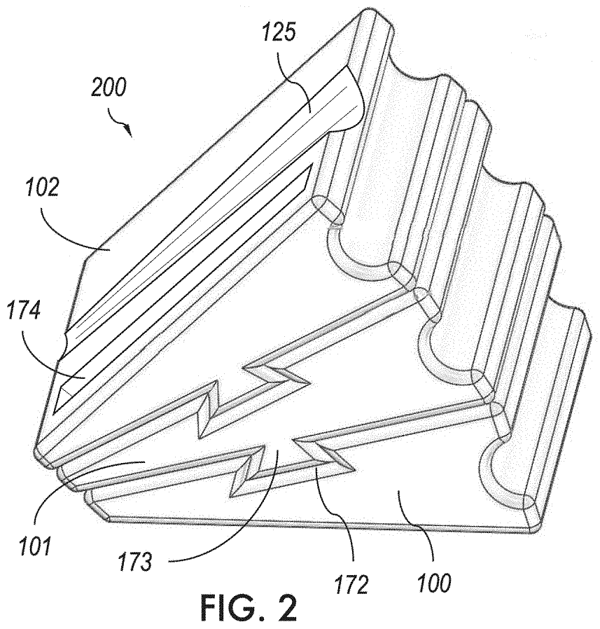

As will be appreciated, in some embodiments, a clinician may need only a single block to maintain a desired support angle between the patient and the medical device. In other embodiments, a clinician may need to stack more than one block to reach the desired support angle. In such embodiments, a first support block may be configured to connect with a second, support block (see, e.g., FIG. 2). For example, as shown in FIG. 1 the support block may have one or more connectors 170, 171, 172 configured to allow the support block to connect with at least one additional support block. In some embodiments, the connectors may be magnetic connectors 170 configured to interact with a magnet or ferromagnetic material 171 that may be on a second support block. In some embodiments, the connectors may be a receiving slot 172 (e.g., a channel) configured to receive a corresponding protruding element from the second support block.

In some embodiments, the support block may have a rear surface 140 that may include a groove 180 configured to provide access to a fingertip.

Referring to FIG. 2, other connectors may be seen. As seen, a system 200 may utilize multiple support blocks 100, 101, 102. In some embodiments, the connector may be a protruding element 173 (here, on support block 101) that is configured to interface with a receiving slot 172 on another support block (here, support block 100).

In some embodiments, the connectors may be one or more protrusions or depressions 174 configured to form an interference fit with one or more protrusions or depressions on a second support block. The connectors also may be hook and loop fasteners, an adhesive (e.g., tape), or another suitable connector or fastener.

As will be appreciated, in some embodiments, each of the stackable blocks may have the same configuration (e.g., shape, angle, height, and/or width). In other embodiments, the stackable blocks may have different configurations (e.g., shape, angle, height, and/or width) such that a clinician may customize the blocks to achieve the desired angle of the medical device exiting the patient.

In some embodiments, the top surface and/or bottom surfaces of the block may be substantially planar. In other embodiments, the top and/or bottom surfaces may not be substantially planar. For example, in some embodiments, one or more blocks may be configured to receive at least a portion of a medical device (or access device). In such embodiments, as shown in FIG. 2, the top surface may include a depression or channel 125 configured to contact and partially surround a portion of the medical device and/or access device.

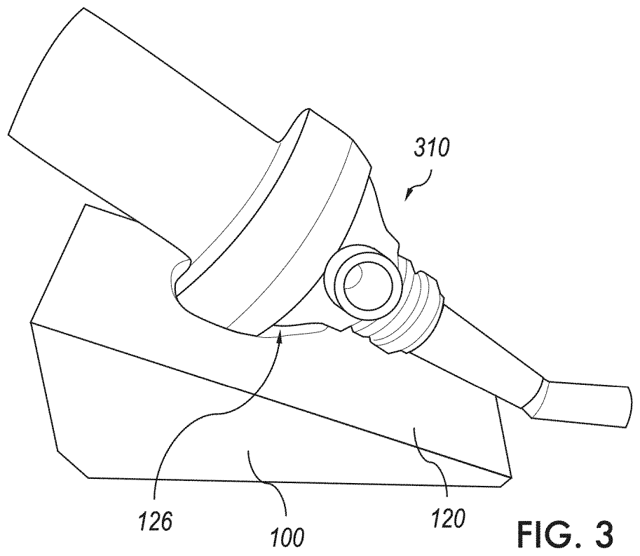

In some embodiments, as shown in FIG. 3, the depressions and/or channels need not extend the entire length of the top surface. For example, as seen in FIG. 3, the top surface 120 may include a depression 126, where the depression contacts and partially surround a portion of an extracorporeal portion of an access device 310. The depression may have a geometric shape. For example, the depression may be circular, semi-hemispherical, triangular, oval, other polygonal or other shape. The depression may have an irregular shape. As shown in FIG. 2, in other embodiments, a channel may extend along an entire length of the block.

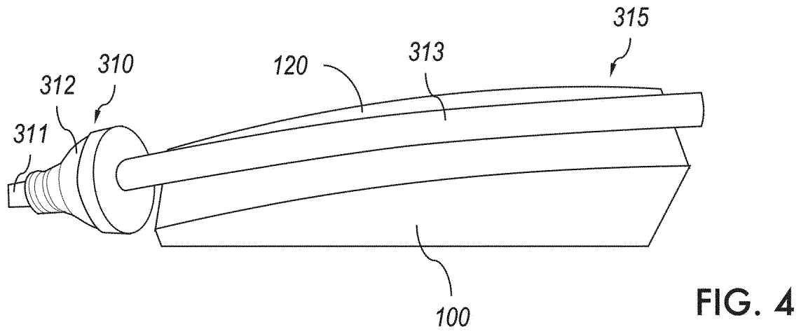

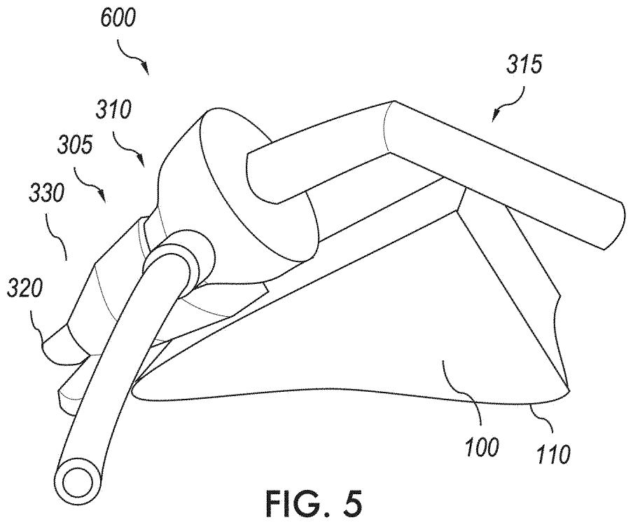

Referring to FIG. 4, in some embodiments, the top surface 120 of the support block may be contoured to match a contour or a medical device (e.g., an extracorporeal portion of a medical device 315). For example, as shown in FIG. 4, the medical device includes a tubular member 313 coupled to a suture hub 314 as well as an access device 310 that includes a sheath hub 312 coupled to a sheath 311. Here, due to the coupling between the tubular member 313 and the sheath hub 312, the support block's support of the tubular member also supports the access device. As shown in FIG. 5, the block may be configured such that a portion of the medical device and/or access device may extend along a portion of the top and/or rear surface.

In some embodiments, as shown in FIG. 5, the bottom surface may also be contoured. For example, in some embodiments, the access device 310 and/or medical device 315 may utilize an access point 320 of a patient 330 where a flat bottom surface is not ideal. In some embodiments, the bottom surface 110 of a support block may be contoured to fit a location on the patient 330 and/or a body type of the patient 330.

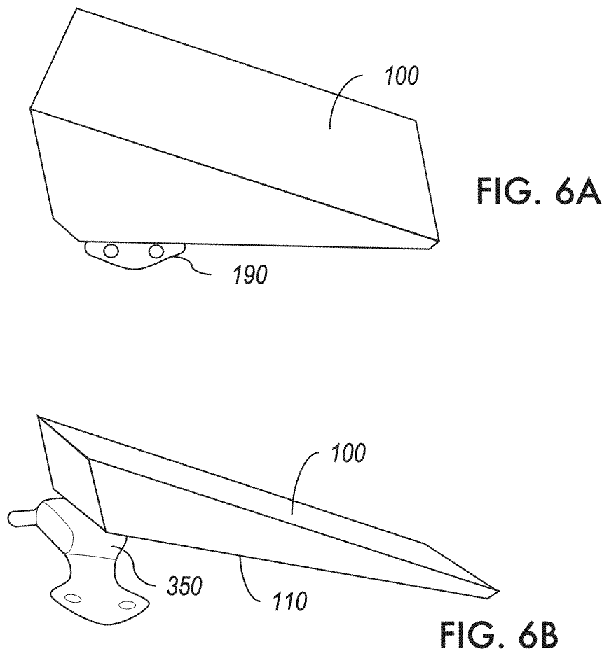

Referring to FIG. 6A, in some embodiments, the support block may be configured such that the block may be sutured to the patient. For example, in some embodiments, the support block 100 may include one or more suture pads 190 configured to allow a user to suture the block to the patient. In some embodiments, the suture pad may be directly coupled to the support block (e.g., adhered, stitched, welded, etc., to a bottom surface). In some embodiments, the suture pad may be formed as part of the support block.

As will be appreciated, in some embodiments, a first block may be suturable to a patient as shown in FIGS. 6A, with one or more blocks being attachable thereto (e.g., as shown in FIG. 2).

As shown in FIG. 6A, in some embodiments, the support block may be attached directly to the suture pad. In other embodiments, as shown in FIG. 6B, the angle of the support block relative to the suture pad may be varied. For example, in some embodiments, an angle-matching device, such as a mini support block, also may be utilized. Referring to FIG. 6B, in some embodiments, an angle-matching device 350 may be coupled to the bottom surface 110 of the support block. As will be appreciated, the support block may be permanently attachable to the angle-matching device or may be removably attachable to the angle-matching device (e.g., via one or more connectors as described herein). In some embodiments, the angle-matching device may be configured to be sutured to the patient. In such embodiments, the angle-matching device may be permanently attached to (e.g., integrally formed with) the support block. In some embodiments, the angle-matching device may be directly coupled to the bottom surface (e.g., adhered, stitched, welded, etc.) of the support block. In some embodiments, the angle-matching device may be indirectly coupled to the bottom surface of the support block.

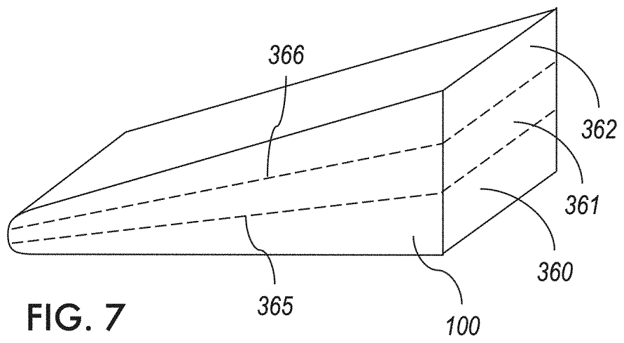

As described herein, the desired support angle of the medical device may be achieved by stacking multiple blocks between the medical device and patient. In other embodiments, as shown in FIG. 7, the desired support angle of the medical device may be achieved via subtractive means rather than additive means. For example, in some embodiments, the support block 100 may include one or more layers 360, 361, 362, where each layer is coupled to the layer below it (e.g., via an adhesive). In such embodiments, the desired support angle of the support block 100 may be set by removing one or more layers 360, 361, 362 from the support block to reduce the initial size of the support block. As with other embodiments, each layer may have a top surface 366 that may be configured to be substantially at an angle (such as 5-30 degrees) relative to the bottom surface 365 of the layer.

In some embodiments, the one or more layers 360, 361, 362 may be adhered together. In other embodiments, the layers may be stitched together, with the layers be removed from one another via cutting the one or more sutures. In still other embodiments, the layers may be formed by creating perforations in a block, e.g., a foam block, such that one layer may be pulled off of the block.

In some embodiments, the support block may include an adhesive layer on a bottom surface, such as for attaching the support block to another support block or to a patient.

In some embodiments, a top surface of the support block may include an adhesive layer, such as for attaching a portion of the medical device or the access device to the support block.

In some embodiments, the support block may include a silicone or a thermoplastic polyurethane. In some embodiments, a support block may include a rigid material. In some embodiments, a support block may include an antiseptic filler. In some embodiments, the antiseptic filler may be silver.

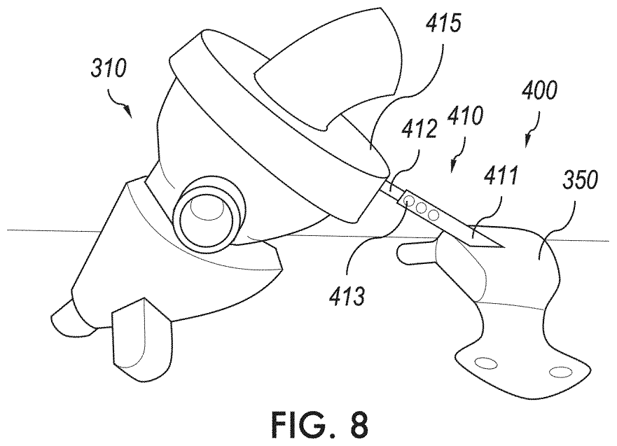

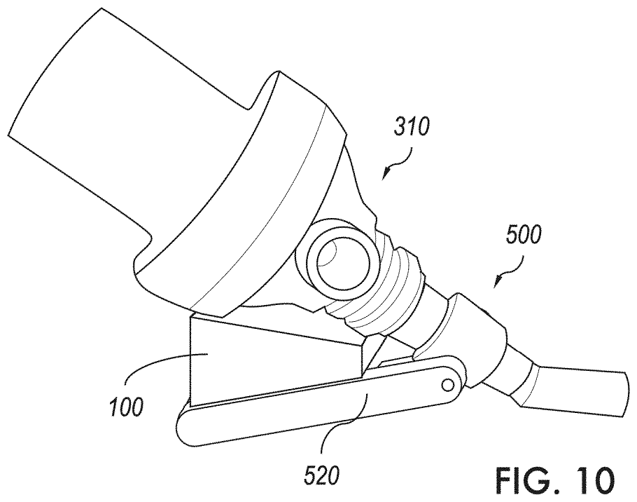

As shown in FIGS. 8-10 and 12-18, the support device may include other structures for achieving the desire support angle between the medical device (or access device) and the patient. For example, in some embodiments, the as shown in FIG. 8, the support device may include a support arm, such as an adjustable arm 410 configured to maintain a desired support angle of the medical device. As shown in this view, the support arm may engage with the access device. For example, in some embodiments, the adjustable arm may be coupled to an access device at or near a proximal end 415 of the access device. In some embodiments, the adjustable arm may be coupled to a sterile hub. In some embodiments, the adjustable arm may be coupled to a sheath hub. As will be appreciated, a length of the support arm may be adjusted to establish the desired support angle of the medical device.

In some embodiments, as shown in FIG. 8, the support arm may engage with a suture pad. In some embodiments, the suture pad also may include an angle-matching device and a medical device, such as an access device, such as a sheath hub, catheter, cannula, and/or stent.

In some embodiments, the adjustable arm 410 may include a lower portion 411 and an upper portion 412. In some embodiments, the upper portion may be slidable relative to the lower portion to adjust the length of the arm. In some embodiments, the upper portion and lower portion may be connected via threads. In some embodiments, the upper portion and lower portion may be connected via a pin button 413.

In some embodiments, the adjustable arm may be a malleable material. The malleable material may be adjustable to a desired position by a user. As will be appreciated, the malleable material also may be configured to hold a desired shape once moved into such a configuration.

In some embodiments, the adjustable arm may be a metal, such as aluminum or stainless steel. In some embodiments, the metal may be annealed. In some embodiments, the metal may be coated with a biologically-compatible material.

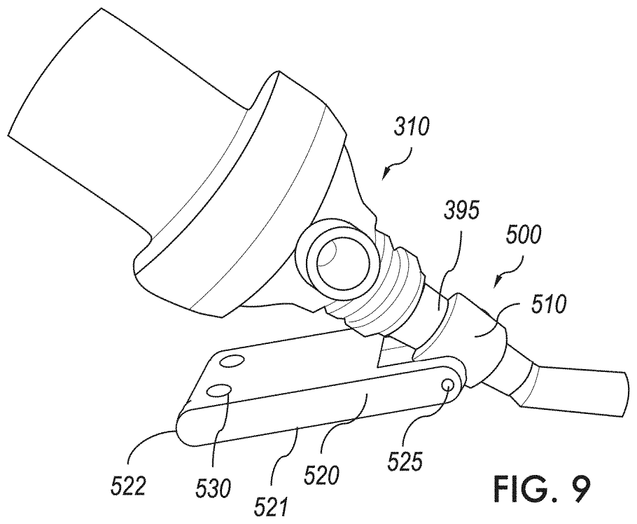

As shown in FIG. 9, in some embodiments, the support device 500 may include a support arm that is attachable to the access device, such as via a collar 510. In some embodiments, the collar may be configured to at least partially surround a hub, catheter, cannula, and/or stent (see e.g., element 395 in FIG. 9) of the access device. As will be appreciated, the collar may be permanently attached to the access device. The collar also may be removably attachable to the access device. In some embodiments, the collar also may be attached to a portion of the medical device (e.g., to the catheter of a blood pump). As shown in FIG. 9, the support arm 520 may be hingedly coupled (e.g., via one or more pins 525) to the collar portion. Although shown as having only a single arm connected to the collar, in other embodiments, more than one support arm may be connected to the collar.

In some embodiments, the collar may be a rigid or semi-rigid material. In some embodiments, the collar may be a flexible material. In some embodiments, the collar may comprise a hook-and-loop fastener system. In some embodiments, the collar may be configured to rotate at least partially around the hub, catheter, cannula, and/or stent 395 of the access device.

In some embodiments, the adjustable arm may include one or more openings 530 to allow the adjustable arm to be sutured to the body of a patient.

In some embodiments, the adjustable arm may be configured such that a side surface 521 of the support arm 520 may contact a patient's body. In some embodiments, the support arm may be configured to have an end portion 522 (e.g., the end of the arm that is furthest from the collar) contact a patient's body.

In some embodiments, the adjustable arm may be removably attached from the collar.

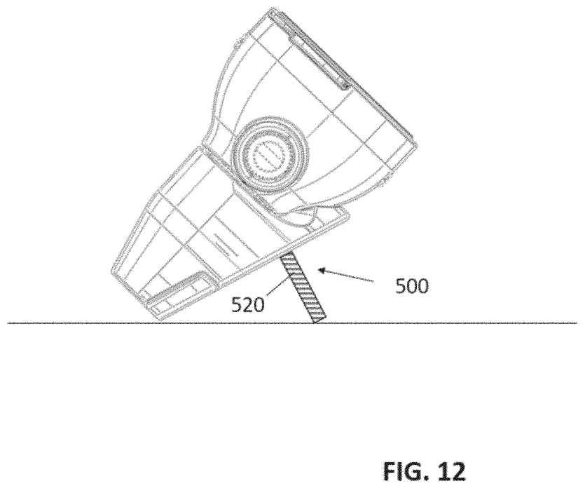

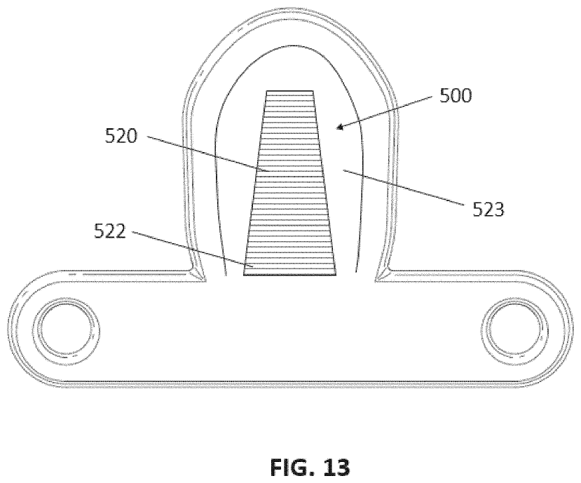

As shown in FIGS. 12 and 13, in some embodiments, the support arm may extend from a rear or a portion of the access device (e.g., a rear portion of an access hub). In such embodiments, the support arm 520 may be stored in a pocket 523 of the rear of the access device. As will be appreciated, such a position may include a storage position of the support arm. As will be further appreciated, the support arm may move to a support position to maintain the desired angle of the access device and/or medical device. In some embodiments, the support arm may be hingedly attached (e.g., via one or more pins) to the access device, with the support arm being pivotable relative to the access device to achieve the desired support angle. As with the above, the support arm may include an end portion 522 that may contact a patient's body. In other embodiments, the support arm may be formed of a flexible material, with the support arm being moveable (e.g., bent) relative to the access device to achieve the desired support angle.

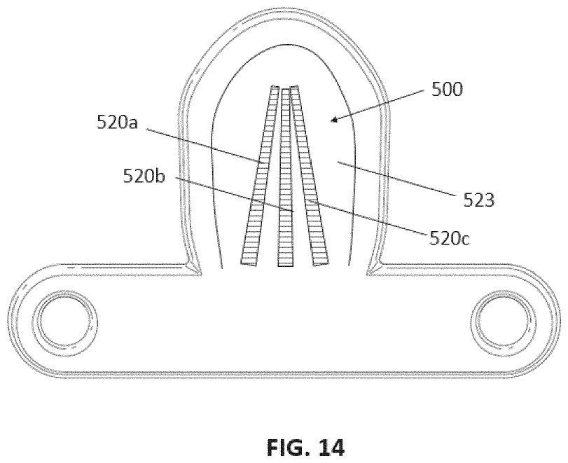

Although shown as having a single support arm, it will be appreciated that the support device may include more than one support arm. For example, as shown in FIG. 14, in some embodiments, the support device may include three support arms 520a, 520b, 520c, which are moveable relative to the access device. As will be appreciated, the support arms may be pivotable relative to the access device to achieve the desired support angle. The support arms also may be bent relative the access device to achieve the desired support angle. As will be appreciated, the support arms may be moved in different directions relative to each other and to the access device to achieve the desired support angle.

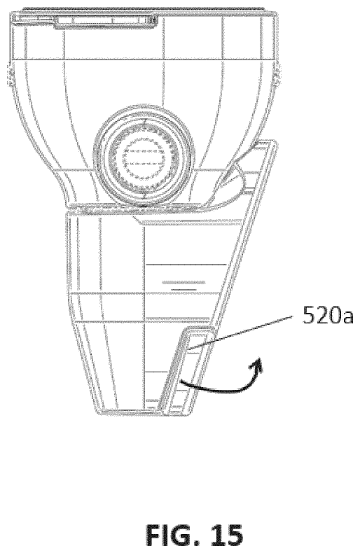

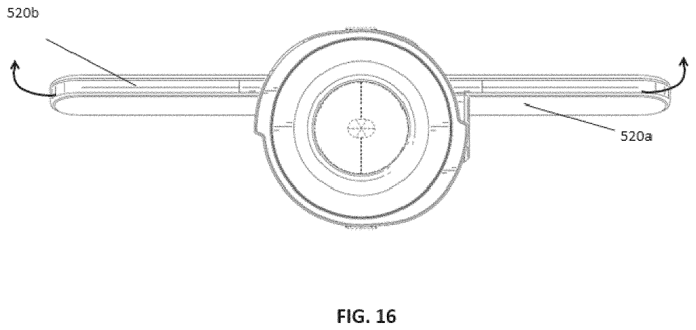

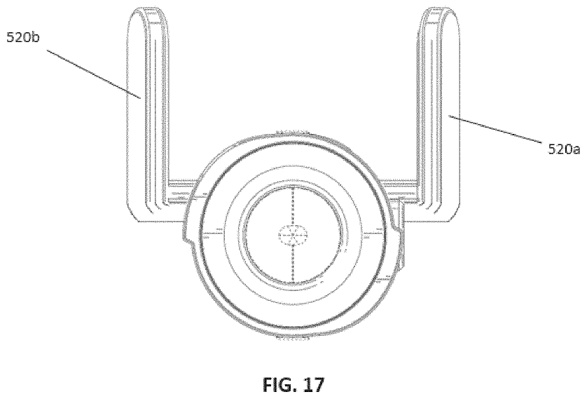

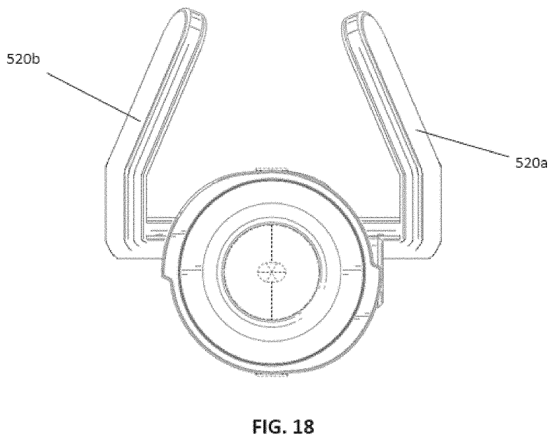

As shown in FIGS. 15-18, the support arms also may extend outwardly from one or more sides of the access device. As shown in FIGS. 16, the first and second arms 520a, 520b, may be parallel to one another. In some embodiments, as shown in FIG. 16, the arms may be moved (e.g., bent) relative to the access device to establish the desired support angle. As shown in FIGS. 17 and 18, the arms may be moved in different arrangements relative to the access device. As will be further appreciated, the arms may be moved in different directions and/or different positions relative to one another to establish the desired support angle. In some embodiments, the support arms may be flexible (e.g., bendable). The support arms also may be pivotable relative to the access device (e.g., if the support arm(s) are attached to the access device via a hinge).

As shown herein, in some embodiments, the support device may include only one or more support blocks or only one or more support arms. In other embodiments, a support device 500 may include both support blocks 100 and support arms 520. In some embodiments, the support block may be placed on the support arm 520 of the support device, and a medical device, such as an access device 310, may be positioned on the support block.

Referencing FIG. 5 again, in some embodiments, a system 600 may be provided. In some embodiments, the system may include an access device 310 having an extracorporeal portion 305 extending away from a patient 330 (e.g., extending out from an access point 320). In some embodiments, the system may include one or more support blocks and/or one or more support arms, each an embodiment of a support block or support arm as disclosed herein (here, support block 100 is shown). The one or more support blocks and/or support arms may be configured to allow the extracorporeal portion 305 to maintain a desired orientation relative to the patient. In some embodiments, the access device may include a catheter. In some embodiments, the access device may be an introducer sheath. In some embodiments, the system may include 2-5 support blocks.

In some embodiments, each support block is configured to have a different angle (e.g., the angle of the top surface relative to the bottom surface). In some embodiments, each support block is configured to have the same angle.

In some embodiments, a bottom surface of one support block may be contoured to fit a location on the patient and/or a body type of the patient. In some embodiments, a top surface of one support block may be contoured or may contain a depression.

In some embodiments, each support block may have a top surface and/or bottom surface that is configured to physically interlock with at least one other support block. In some embodiments, each support block may have a top surface and/or bottom surface that is configured to interact magnetically with at least one other support block. In some embodiments, each support block may have a top surface and/or bottom surface that is configured to connect to at least one other support block via an interference fit.

In some embodiments, a kit is provided. The kit may include one or more support blocks, each independently an embodiment of a support block as disclosed herein. The one or more support blocks may be configured to allow an extracorporeal portion of the access device (and, thus, the medical device) to maintain a desired orientation relative to a patient, each support block comprising a bottom surface and a top surface substantially at an angle relative to the bottom surface. The kit may include a suture pad (with or without an angle-matching device) coupled to a bottom surface of one of the one or more support blocks, the angle-matching device configured to be sutured to the patient. In some embodiments, the kit also may include an access device (e.g., a catheter or introducer sheath).

In some embodiments, the kit may include 2-5 support blocks. In some embodiments, each support block is configured to have the same angle. In some embodiments, each support block is configured to have a different angle.

In some embodiments, a bottom surface of one support block may be contoured to fit a location on the patient and/or a body type of the patient. In some embodiments, a top surface of one support block may be contoured or may contain a depression.

In some embodiments, each support block may have a top surface and/or bottom surface that is configured to physically interlock with at least one other support block. In some embodiments, each support block may have a top surface and/or bottom surface that is configured to interact magnetically with at least one other support block. In some embodiments, each support block may have a top surface and/or bottom surface that is configured to connect to at least one other support block via an interference fit.

In some embodiments, the kit may include one or more support arms that are engageable with an access device.

In some embodiments, the kit may include an access device having one or more support arms. In some embodiments, the kit may include a suture pad (with or without an angle-matching device).

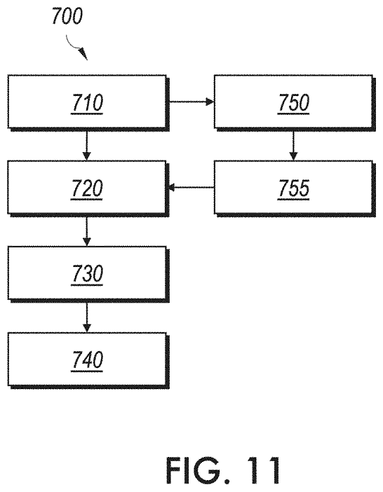

In some embodiments, a method may be provided. Referring to FIG. 11, the method 700 may include inserting 710 a medical device into a patient such as via an access device. In such embodiments, an extracorporeal portion of the access device and/or medical device may extend away from the skin of the patient. The method 700 may also include placing 720 one or more support blocks and/or support devices on the skin of the patient, under the extracorporeal portion, and allowing the extracorporeal portion to rest at least partially on the support block and/or device.

The support blocks and/or support devices may each independently be an embodiment of a support block or support device as disclosed herein.

In some embodiments, the method may include securing 730 the access device to at least one support block and/or support device. This may be done via sutures, adhesive, tape, or other appropriate means of attachment.

In some embodiments, the method may include securing 740 at least one support block and/or support device to the surface of the patient.

In some embodiments, the method may include removably coupling 750 a first support block to a second support block or a first support device. In some embodiments, it is performed prior to the placing 720 of the support blocks on the surface of the patient. In some embodiments, it may be done at any time. In some embodiments, the method may include coupling 755 an angle-matching device to a bottom surface of a support block or support device, the angle-matching device configured to be sutured to the patient.

Embodiments of the present disclosure are described in detail with reference to the figures wherein like reference numerals identify similar or identical elements. It is to be understood that the disclosed embodiments are merely examples of the disclosure, which may be embodied in various forms. Well known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

Those skilled in the art will recognize or be able to ascertain using no more than routine experimentation many equivalents to the specific embodiments of the invention described herein. Such equivalents are intended to be encompassed by the following claims.

1. A support block, comprising:
a bottom surface;
a top surface substantially at an angle relative to the bottom surface, the angle being between 5 degrees and 50 degrees, the top surface connected to the bottom surface via a front surface, two side surfaces, and a rear surface, the rear surface having a larger surface area than the front surface;
wherein the top surface and/or bottom surface has a connector configured to allow the support block to connect with at least one additional support block, the connector being:
a protruding element configured to interface with a receiving slot on the at least one additional support block;
a receiving slot configured to receive a protruding element from the at least one additional support block;
a magnet configured to interface with a magnet or ferromagnetic material on the at least one additional support block; and/or
one or more protrusions or depressions configured to form an interference fit with one or more protrusions or depressions on the at least one additional support block.

2. The support block according to claim 1, wherein support blocks comprise a silicone, a thermoplastic polyurethane, and/or a foam.

3. The support block according to claim 1, wherein the one or more support blocks comprise a rigid material.

4. The support block according to claim 1, wherein the one or more support blocks comprise an antiseptic filler.

5. The support block according to claim 4, wherein the antiseptic filler comprises silver or chlorohexidine gluconate (CHG).

6. The system according to claim 1, further comprising an angle-matching device coupled to the bottom surface, the angle-matching device configured to be sutured to the patient.

7. The system according to claim 1, wherein the support block is configured to be sutured to the patient.

8. The system according to claim 1, further comprising an adhesive layer on a bottom surface and/or a tacky surface on the bottom surface of the support block.

9. The system according to claim 1, wherein the top surface is contoured to receive at least a portion of an access device and/or a medical device.

10. The support block according to claim 9, wherein the top surface comprises a depression configured to receive at least a portion of the access device and/or the medical device.

11. The system according to claim 1 wherein the bottom surface of one support block is contoured to fit a location on the patient and/or a body type of the patient.

12. The system according to claim 1, wherein a rear surface comprises a groove configured to provide access to a fingertip.

13-21. (canceled)

22. A system comprising:
a medical device and/or an access device having an extracorporeal portion extending away from a patient; and
one or more support devices, the one or more support structures comprising:
one or more support blocks configured to allow the extracorporeal portion to maintain a desired support angle relative to the patient, each support block comprising a bottom surface and a top surface substantially at an angle relative to the bottom surface; and/or
one or more support arms.

23-34. (canceled)

35. The system according to claim 22, wherein each support block has a top surface and/or bottom surface that is configured to physically interlock with at least one other support block.

36. The system according to claim 22, wherein each support block has a top surface and/or bottom surface that is configured to interact magnetically with at least one other support block.

37. The system according to claim 22, wherein each support block has a top surface and/or bottom surface that is configured to connect to at least one other support block via an interference fit.

38. The system according to claim 22, wherein the one or more support blocks comprises 2-5 blocks.

39. The system according to claim 22, wherein the angle is between 5 degrees and 30 degrees.

40-42. (canceled)

43. The system according to claim 22, wherein, for each support block, the top surface is connected to the bottom surface via a front surface, two side surfaces, and a rear surface, the rear surface having a surface area greater than a surface area of the front surface, and the rear surface comprising a groove configured to provide access to a fingertip.

44-64. (canceled)

65. A method for supporting an access device, comprising:
inserting an access device into a patient, such that an extracorporeal portion of the access device extends away from a surface of the patient; and
placing one or more support structures on the surface of the patient, under the extracorporeal portion, and allowing the extracorporeal portion to rest at least partially on the support structure, the one or more support structures comprising:
one or more blocks configured to allow the extracorporeal portion to maintain a desired orientation relative to the patient, each support block comprising a bottom surface and a top surface substantially at an angle relative to the bottom surface; and/or
one or more support devices, each support device comprising an adjustable arm.

66-88. (canceled)
